(19) United States
(12) Patent Application Publication
(10) Pub. No.: US 2005/0177200 A1
(43) Pub. Date: Aug. 11, 2005

(54) Method, apparatus and system for determining effects and optimizing parameters of vagus nerve stimulation

(76) Inventors: Mark S George, Sullivans Island, SC (US); Daryl E. Bohning, Warren, CT (US); Ziad Nahas, Charlston, SC (US)

Correspondence Address: NEEDLE & ROSENBERG, P.C., SUITE 1000, 999 PEACHTREE STREET, ATLANTA, GA, 30309-3915, US

(21) Appl. No.: 10/513,168

(22) PCT Filed: May 5, 2003
(86) PCT No.: PCT/US03/14245

Related U.S. Application Data
(60) Provisional application No. 60/377,692, filed on May 3, 2002.

(51) Int. Cl. A61N 1/18

(52) U.S. Cl. 607/45

(57) ABSTRACT

Effects of vagus nerve stimulation (VNS) on, for example, regions of the brain, are determined by synchronizing application of the VNS with functional magnetic resolution imaging (fMRI) scanning (FIG. 1). Parameters of the VNS application may be optimized using the fMRI images. Optimal dosage and protocols for the VNS application may be set based on the determined effects of VNS application on regional brain activity. Also, a blood oxygenation level-dependent (BOLD) response of one or more brain regions to VNS application may be determined based on the fMRI images. VNS may also be used to treat neuropsychiatric diseases by mapping effects or VNS application on brain regions u) regional effects or the neuropsychiatric disease. The change in effects of VNS application on the patient with continued use may be assessed based on long-term and repeated studies of effects of VNS application.

### CROSS-REFERENCE TO RELATED APPLICATION

This application claims priority to U.S. Provisional Application No. 60/377,692, herein incorporated by reference.

### BACKGROUND

The present invention generally relates to vagus nerve stimulation. More particularly, the present invention relates to a method, apparatus, and system for determining effects and optimizing parameters of vagus nerve stimulation by interleaving vagus nerve stimulation with functional magnetic resonance imaging.

Vagus nerve stimulation (VNS) has shown beneficial clinical effects in treating epilepsy and has shown promise in treating patients with major depressions. VNS involves applying mild, intermittent electrical impulses to the vagus nerve of the human neck. Little is known about the mechanism responsible for VNS effects. Researchers have typically applied the maximum tolerable intensity while other parameters of the stimulation, such as frequency, pulse width, and stimulation duration, are held constant. Multi-parameter optimizations of the technique have been largely unexplored. Clinical trials in epilepsy have shown a pattern of progressive efficacy over time.

Positron emission tomography (PET) has been used to investigate the effects of VNS. Unfortunately, the low temporal resolution of PET limits its observations to integrated effects over time periods longer than typical VNS stimulation. Further, the dependence of PET methods on the use of radioactive tracers is unsuitable for repeated use on a single patient.

Therefore, there is a need to develop a technique and apparatus for determining effects of VNS and optimizing parameters of VNS so as to improve the performance and applications of VNS.

### SUMMARY

According to exemplary embodiments, a method, system and apparatus are provided for performing VNS-synchronized functional magnetic resonance imaging (fMRI to determine effects and optimize parameters of VNS application on a patent.

According to one embodiment, a computer is used to detect the electrical impulses generated by a VNS stimulator and synchronize fMRI image acquisition with the VNS impulses.

VNS-fMRI may be used to determine the effects of the many VNS parameters on regional brain activity to help set optimal dosage and protocols in clinical use of VNS.

VNS-synchronized fMRI may also be used study the blood oxygenation level-dependent (BOLD) response of several brain regions to VNS stimulation. The VNS responses of the orbitofrontal cortex, parieto-occipital cortex, left temporal cortex, hypothalamus, and left amygdala regions of the human brain can be determined.

Further, VNS-fMRI may be used to determine useful VNS applications in the treatments of neuropsychiatric diseases. The mappings of VNS effects on brain regions are considered in relation to the regional effects of such diseases.

Also, because of its safe, noninvasive nature, VNS-fMRI provides for long-term and repeated studies of VNS treatment to assess whether regional brain effects change with continued use.

These and other aspects will become apparent from the following description of various embodiments taken in conjunction with the Appendices, although variations and modifications may be effected without departing from the spirit and scope of the novel concepts of the disclosure.

### BRIEF DESCRIPTION OF THE DRAWINGS

FIG. 1 is a schematic diagram showing a system for vagus nerve stimulation and functional magnetic resonance imaging according to an exemplary embodiment.

### DETAILED DESCRIPTION

Several embodiments of the invention are now described in detail in connection with the disclosures made in the Appendices. As used in the description herein and throughout the claims that follow, the meaning of “a,” “an,” and “the” includes plural reference unless the context clearly dictates otherwise. Also, as used in the description herein and throughout the appendices that follow, the meaning of “in” includes “in” and “on” unless the context clearly dictates otherwise.

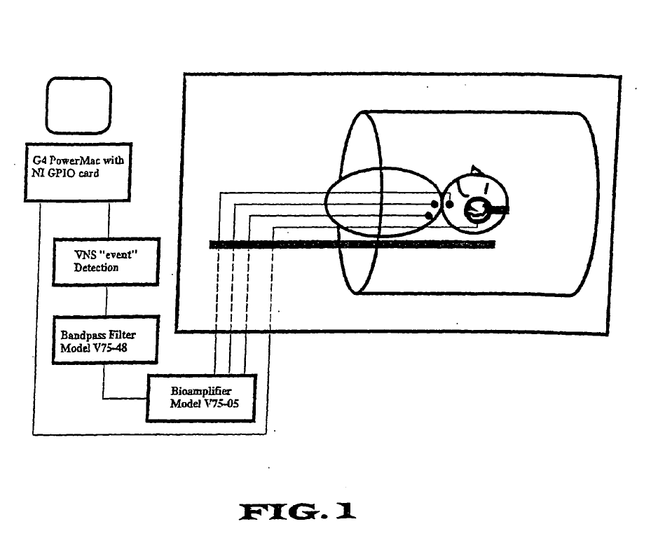

An exemplary system for synchronizing VNS application with fMRI is schematically shown in FIG. 1.

Referring to FIG. 1, the signal from an implanted VNS generator is monitored with an external computer to determine the exact timing of the VNS cycle. An auditory signal reference stimulus is provided through headphones to the patient, and blood oxygenation level-dependent fMRI images are collected. VNS parameters that the patient cannot detect but that have effects in various brain regions are obtained and analyzed.

In one embodiment, a VNS generator is implanted within the neck of a human subject in an MRI-compatible fashion with the lead pins oriented along the long axis of the body. Adhesive-backed, MRI-compatible electrodes and electrocardiogram leads are placed on the subject's neck. At least one electrode is placed over the route of the implanted VNS leads just above and/or below the incision scar. An additional electrode is placed about 3 to 4 cm posterior to the scar to serve as an electrical ground. The subject is given earphones and instructed to lie quietly with eyes closed and to listen for an auditory tone. The head of the subject is stabilized within the scanner head coil with foam-padded adjustable restraints. The VNS pulses are detected by the electrodes and analyzed to achieve synchronization with fMRI scanning.

A patient's VNS device may be programmed to a predetermined time cycle such as a 7-seconds-on/108-seconds-off stimulation cycle. This cycle is the shortest stimulation duration setting of some available devices and best delineates the time course of the VNS response. During the fMRI scan procedure, a 440-Hz tone is fed through the headphones in 7-second trains of 100-ms pulses.

In some embodiments, structural fMRI images are transferred to a computing platform and stored in a memory device for anatomic reference. A check can be performed to ensure that the subject movement during scanning is within acceptable limits. The images can be motion corrected when necessary. Images can be spatially normalized to match standard brain template configurations with an affine transformation. A high-pass filter can be utilized to remove signal drift, cardiac and respiratory effects, and other low-frequency artifacts.

According to one embodiment, a determination of neuropsychiatric diseases that VNS might treat, may be made by outlining the neurobiologic effects of VNS, listing the functional neuroanatomic maps and pathophysiological cascades of neuropsychiatric diseases, identifying overlaps between the mappings of VNS effects and neuropsychiatric diseases, and carrying out preclinical and clinical trials in those diseases which show high probabilities of VNS therapeutic effects.

In one embodiment, an automated shimming apparatus, system and method allow real-time analysis in determining the best VNS device settings based on pre-determined regional brain activation. A VNS generator is provided which can be variably programmed to enable the use of single event fMRI to efficiently explore VNS parameters. Remote programming of the VNS generator allows shimming of the generator settings so that maximum brain effect settings can be determined.

Computer software may be used to determine the exact time of each VNS pulse. This provides images that are sensitive to the small changes that occur in areas of the brain activated by VNS. This involves averaging a series of images that follow VNS pulses in lock-step fashion and associating the time course of brain activity with a single event, such as a sensory stimulation, a movement, or a thought.

More details of the present invention are further disclosed in the Appendices including all texts and drawings therein, which form an integral part of the present application.

While various embodiments of the invention are described above and in the Appendices, it is to be understood that certain changes can be made in the form and arrangement of the elements of each system and steps of each method according to the present invention as would be known to one skilled in the art without departing from the underlying scope of the invention as is particularly described above including the Appendices. Furthermore, the embodiments described above are only intended to illustrate the principles of the present invention and are not intended to limit the invention to the disclosed elements.

### APPENDICES

Appendix A, total pages 10, A1-A10,
Appendix B, total pages 9, B1-B9,
Appendix C, total pages 15, C1-C15,
Appendix D, total pages 15, D1-D15,
Appendix E, total pages 27, E1-E27,
Appendix F, total pages 6, F1-F6,
Appendix G, total pages 9, G1-G9.

1. A method for determining the effects of vagus nerve stimulation, comprising:
detecting electrical impulses applied to stimulate the vagus nerve of a patient; and
synchronizing operation of a functional magnetic resolution image (FMRI) scanner with the detected electrical impulses, wherein images produced by the fMRI scanner indicate effects of applying vagus nerve stimulation (VNS) on the patient.

2. The method of claim 1, further comprising performing scanning by the fMRI scanner in synchronization with application of VNS.

3. The method of claim 1, further comprising using the images produced by the fMRI scanner to determine effects of VNS application on regional brain activity.

4. The method of claim 3, further comprising setting optimal dosage and protocols for VNS application based on the determined effects of VNS application on regional brain activity.

5. The method of claim 1, further comprising determining a blood oxygenation level-dependent (BOLD) response of one or more brain regions to VNS application based on the images produced by the fMRI scanner.

6. The method of claim 3, wherein the images are used to determine the effects of VNS application on at least one of the orbitofrontal cortex, parieto-occipital cortex, left temporal cortex, hypothalamus, and left amygdala regions of the brain.

7. The method of claim 1, further comprising using VNS application to treat at least one neuropsychiatric disease, based on the determined effects of VNS application.

8. The method of claim 7, wherein the step of using VNS application to treat the neuropsychiatric disease comprises mapping effects of VNS application on brain regions to regional effects of the neuropsychiatric disease.

9. The method of claim 1, further comprising optimizing application of the VNS based on the fMRI images generated by the scanner.

10. The method of claim 9, wherein the step of optimizing includes optimizing at least one of intensity, frequency pulse width, and duration of the electrical impulses based on the fMRI images.

11. The method of claim 1, further comprising assessing whether of effects of VNS application on the patient change with continued use based on long-term and repeated studies of effects of VNS application.

12. An apparatus for determining the effects of vagus nerve stimulation, comprising: means for detecting electrical impulses applied to stimulate the vagus nerve of a patient; and means for synchronizing operation of a functional magnetic resolution image (fMRl) scanner with the detected electrical impulses, wherein images produced by the fMRI scanner indicate effects of vagus nerve stimulation (VNS) on the patient.

13. The apparatus of claim 12, wherein scanning is performed by the fMRI scanner in synchronization with application of VNS.

14. The apparatus of claim 12, wherein the images produced by the FMRI scanner are used to determine effects of VNS application on regional brain activity.

15. The apparatus of claim 14, wherein optimal dosage and protocols for VNS application are set based on the determined effects of VNS application on regional brain activity.

16. The apparatus of claim 12, wherein a blood oxygenation level-dependent (BOLD) response of one or more brain regions to VNS application is determined based on the images produced by the fMRI scanner.

17. The apparatus of claim 14, wherein the images are used to determine the effects of VNS on at least one of the orbitofrontal cortex, parieto-occipital cortex, left temporal cortex, hypothalamus, and left amygdala regions of the brain.

18. The apparatus of claim 12, wherein VNS application is used to treat at least one neuropsychiatric disease, based on the determined effects of VNS application.

19. The apparatus of claim 18, wherein VNS application is used to treat the neuropsychiatric disease by mapping effects of VNS on brain regions to regional effects of the neuropsychiatric disease.

20. The apparatus of claim 12, wherein application of the VNS is optimized based on the fMRI images generated by the scanner.

21. The apparatus of claim 12, wherein at least one of intensity, frequency, pulse, width and duration of the applied electrical impulses to stimulate the vagus nerve is optimized based on the fMRI images.

22. The apparatus of claim 12, wherein a change in the effects of VNS application on the patient is assessed based on long-term and repeated studies of effects of VNS application.

23. A system for determining the effects of vagus nerve stimulation, comprising: a generator for generating electrical impulses and applying the impulses to stimulate the vagus nerve of a patient; a functional magnetic resolution image (FMRI) scanner for producing a magnetic image of the patient; and a processor for synchronizing operation of the fMRI scanner with the detected electrical impulses such that images produced by the fMRI scanner indicate effects of application of vagus nerve stimulation (VNS) on the patient.

24. The system of claim 23, wherein the fMRI scanner produces magnetic images of the patient in synchronization with application of VNS.

25. The system of claim 23, wherein the images produced by the FMRI scanner are used to determine effects of VNS application on regional brain activity.

26. The system of claim 25, wherein optimal dosage and protocols for VNS application are set based on the determined effects of VNS application on regional brain activity.

27. The system of claim 23, wherein a blood oxygenation level-dependent (BOLD) response of one or more brain regions to VNS application is determined based on the images produced by the fMRI scanner.

28. The system of claim 25, wherein the images are used to determine the effects of VNS application on at least one of the orbitofrontal cortex, parieto-occipital cortex, left temporal cortex, hypothalamus, and left amygdala regions of the brain.

29. The system of claim 23, wherein VNS application is used to treat at least one neuropsychiatric disease, based on the determined effects of VNS application.

30. The system of claim 29, wherein VNS application is used to treat the neuropsychiatric disease by mapping effects of VNS on brain regions to regional effects of the neuropsychiatric disease.

31. The system of claim 23, wherein application of VNS is optimized based on the fMRI images produced by the scanner.

32. The system of claim 31, wherein at least one of intensity, frequency, pulse width and duration of the electrical impulses applied to stimulate the vagus nerve are optimized based on the fMRI images.

33. The system of claim 23, wherein a change of effects of VNS on the patient with continued use is assessed based on long-term and repeated studies of effects of VNS.
